(19) United States
(12) Patent Application Publication
(10) Pub. No.: US 2026/0014104 A1
(43) Pub. Date: Jan. 15, 2026

(54) COMPOSITIONS AND METHODS FOR TREATING CYSTITIS

(71) Applicant: FERRING B.V., Hoofddorp (NL)

(72) Inventors: Bijay Kumar PADHI, Hyderabad (IN); Ankit BAHETI, Hyderabad (IN); Sandeep Suresh ZODE, Hyderabad (IN)

(73) Assignee: FERRING B.V., Hoofddorp (NL)

(21) Appl. No.: 19/147,222

(22) PCT Filed: Jan. 12, 2024
(86) PCT No.: PCT/IB2024/050334

§ 371 Date: Jul. 10, 2025

(30) Foreign Application Priority Data
Jan. 13, 2023 (IN) 202311002763

(51) Int. Cl. A61K 31/167; A61K 9/08; A61K 31/728; A61K 31/737; A61K 47/26

(52) U.S. Cl. CPC A61K 31/167; A61K 9/08; A61K 31/728; A61K 31/737; A61K 47/26

(57) ABSTRACT

Described herein are aqueous pharmaceutical compositions comprising lidocaine, hyaluronic acid, and chondroitin sulphate (or a pharmaceutically acceptable salts of any thereof) and water, as well as methods of making such aqueous pharmaceutical compositions, and therapeutic methods using them.

### CROSS-REFERENCE TO RELATED APPLICATIONS

This application claims priority to Indian patent application Number 202311002763, filed Jan. 13, 2023, the entire contents of which are incorporated herein by reference.

### FIELD

Described herein are aqueous pharmaceutical compositions comprising, e.g., lidocaine, hyaluronic acid, and chondroitin sulphate, as well as methods of making such aqueous pharmaceutical compositions, and therapeutic methods using them.

### BACKGROUND

Cystitis is a condition of inflammation of the bladder. Cystitis may be caused by a bladder infection, chemotherapy, or radiation treatment. Interstitial cystitis (IC) is a progressive bladder disorder with symptoms including mild to severe bladder pain and an urgent and/or frequent need to urinate. The disease etiology remains uncertain; it is postulated that there is an initial infective insult that damages the glycosaminoglycan (GAG) layer of the bladder urothelium. Breakdown of the protective GAG layer may allow potentially harmful substances in urine (e.g., solutes or ions such as potassium) to reach the bladder wall, causing pain and discomfort. See, C.L. Parsons, Rev. Urol. 4 (Supp. 1: S49-S55 (2002).

Currently treatments for cystitis are primarily targeted to control symptoms. First-line treatments may include behavioral and diet changes, psychological stress management, urogynecological exercises, and heat or cold therapy. Second-line treatments may include oral and intravesical medication targeting pain and/or GAG replenishing agents. Oral pentosan polysulfate is the only oral medication currently approved for treating cystitis. Chondroitin sulphate sodium and sodium hyaluronate are the most widely used GAG replenishing agents, either alone or in combination. Heparin and pentosan polysulfate also may be used to heal the GAG layer of the bladder. Dimethyl sulfoxide (DMSO) is indicated for symptomatic relief. Lidocaine may be used off label intravesically to relieve bladder pain. See, e.g., Garzon et al., Menopause Review/Przegląd Menopauzalny 19: 35-43 (2020).

Thus, treatment with a combination of a GAG replenishing agent (e.g., chondroitin sulphate sodium and/or sodium hyaluronate) and a local anaesthetic (e.g., lidocaine) may be useful for reducing acute pain and promoting healing of the bladder wall GAG layer in subjects suffering from cystitis, including IC. However, no such compositions have been approved for pharmaceutical use. DE202017104675 and EP3415163 disclose compositions comprising lidocaine and a GAG replenishing agent such as chondroitin sulphate sodium and sodium hyaluronate. The disclosed compositions include appreciable amounts of buffering agents, which can be disadvantageous in the target patient population because the bladder wall of a cystitis patient is susceptible to certain solutes and ions. Thus, exposure to such buffer components may cause the patient irritation and pain.

Thus, there remains a need for pharmaceutical compositions suitable for use in treating cystitis, including pharmaceutical compositions comprising, e.g., lidocaine, hyaluronic acid, and chondroitin sulphate.

### SUMMARY

Provided herein are aqueous pharmaceutical compositions comprising lidocaine, hyaluronic acid, and chondroitin sulphate (or pharmaceutically acceptable salts of any thereof), and water, as well as methods of making such aqueous pharmaceutical compositions, and therapeutic methods using them.

In accordance with one aspect, there are provided aqueous pharmaceutical compositions comprising (i) 0.05% to 0.6% w/v of lidocaine or a pharmaceutically acceptable salt thereof; (ii) 0.25% to 3.0% w/v of hyaluronic acid or a pharmaceutically acceptable salt thereof; (iii) 0.3% to 3.5% w/v of chondroitin sulphate or a pharmaceutically acceptable salt thereof, and (iv) a pH adjusting agent in an amount effective to achieve a pH of the composition of from 6.0 to 8.5, wherein the composition does not include a buffering agent in an amount effective to buffer the pH of the composition.

In some embodiments, the composition does not include one or more or all of the following buffering agents: a phosphate salt, a carbonic acid, a bicarbonate salt, an acetic acid, an acetate salt, a silicate salt, a citric acid, and a citrate salt. In some embodiments, the composition does not include any of the following buffering agents: a phosphate salt, a carbonic acid, a bicarbonate salt, an acetic acid, an acetate salt, a silicate salt, a citric acid, and a citrate salt.

In some embodiments, the hyaluronic acid or pharmaceutically acceptable salt thereof, has a weight-average molecular weight of from about 50 to about 500 kDa. In some embodiments, the hyaluronic acid or pharmaceutically acceptable salt thereof is sodium hyaluronate.

In some embodiments, the chondroitin sulphate or pharmaceutically acceptable salt thereof has a weight-average molecular weight of from about 1 to about 200 kDa. In some embodiments, the chondroitin sulphate or pharmaceutically acceptable salt is chondroitin sulphate sodium.

In some embodiments, the lidocaine or pharmaceutically acceptable salt thereof is lidocaine hydrochloride.

In some embodiments, the pH adjusting agent is selected from meglumine and NaOH.

In some embodiments, the composition comprises (i) 0.4% w/v of lidocaine hydrochloride, (ii) 1.6% w/v of sodium hyaluronate, (iii) 2% w/v of chondroitin sulphate sodium, and (iv) a pH adjusting agent selected from meglumine and NaOH.

The composition may comprise about 4 mg/mL lidocaine hydrochloride, about 16 mg/ml sodium hyaluronate, and about 20 mg/mL chondroitin sulphate sodium (CS).

The composition may be packaged in a unit dose form comprising 200 mg lidocaine hydrochloride in 50 mL.

In some embodiments, the composition comprises (i) 0.2% w/v of lidocaine hydrochloride, (ii) 0.8% w/v of sodium hyaluronate, (iii) 1% w/v of chondroitin sulphate sodium, and (iv) a pH adjusting agent selected from meglumine and NaOH.

The composition may comprise about 2 mg/mL lidocaine hydrochloride, about 8 mg/mL sodium hyaluronate, and about 10 mg/mL chondroitin sulphate sodium (CS).

The composition may be packaged in a unit dose form comprising 100 mg lidocaine hydrochloride in 50 mL.

In any embodiments, the composition may have a pH from 7.3 to 7.5, such as a pH of about 7.4.

In any embodiments, the composition may comprise an amount of pH adjusting agent effective to adjust the pH to about 7.4.

In any embodiments, the composition may be a sterile solution formulated for instillation in the urogenital tract or bladder.

In any embodiments, the composition may be stable for at least 3 months when stored at 25° C. and 60% relative humidity. After storage for 3 months at 25° C. and 60% relative humidity, the amount of lidocaine degradation products in the composition may be less than 0.4 wt %. The composition may be stable for at least 3 months when stored at 30° C. and 75% relative humidity. After storage for 3 months at 30° C. and 75% relative humidity, the amount of lidocaine degradation products in the composition may be less than 0.4 wt %. The composition may be stable for at least 3 months when stored at 40° C. and 75% relative humidity. After storage for 3 months at 40° C. and 75% relative humidity, the amount of lidocaine degradation products in the composition may be less than 0.5 wt %. After storage for 3 months at 25° C. and 60% relative humidity, the pH of the composition may differ by no more than about 0.3 from the original pH.

Also provided are methods of treating cystitis, comprising administering a pharmaceutical composition as described herein to a subject in need thereof. The pharmaceutical composition may be administered by instillation into the urogenital tract or bladder. The subject may have a reduction in nociceptive pain score after administration of the pharmaceutical composition. The subject may have a lower bladder pressure after administration of the pharmaceutical composition. The cystitis treated may be interstitial cystitis.

Also provided are an aqueous pharmaceutical composition in accordance with any embodiments described herein for use in treating cystitis. The cystitis may be interstitial cystitis.

Also provided are uses of an aqueous pharmaceutical composition in accordance with any embodiments described herein for the manufacture of a medicament for treating cystitis. The cystitis may be interstitial cystitis.

### BRIEF DESCRIPTION OF THE DRAWINGS

FIG. 1 illustrates the stability (with respect to lidocaine degradation products) of aqueous pharmaceutical compositions as described in Table 1 under various conditions (25° C. and 60% relative humidity, 30° C. and 75% relative humidity, and 40° C. and 75% relative humidity, respectively).

FIG. 2 illustrates the stability (with respect to pH) of aqueous pharmaceutical compositions as described in Table 1 under various conditions (25° C. and 60% relative humidity, 30° C. and 75% relative humidity, and 40° C. and 75% relative humidity, respectively).

FIG. 3 illustrates the stability (with respect to lidocaine degradation products) of aqueous pharmaceutical compositions as described in Table 2 under various conditions (25° C. and 60% relative humidity, 30° C. and 75% relative humidity, and 40° C. and 75% relative humidity, respectively).

FIG. 4 illustrates the stability (with respect to pH) of aqueous pharmaceutical compositions as described in Table 2 under various conditions (25° C. and 60% relative humidity, 30° C. and 75% relative humidity, and 40° C. and 75% relative humidity, respectively).

### DETAILED DESCRIPTION

As noted above, provided herein are aqueous pharmaceutical compositions comprising lidocaine, hyaluronic acid, and chondroitin sulphate (or pharmaceutically acceptable salts of any thereof), and water, as well as methods of making such aqueous pharmaceutical compositions, and therapeutic methods using them, including for treating cystitis (including IC). The disclosed compositions do not rely on a buffer to maintain pH, and so avoid the risks a buffer may post to cystitis patients.

Lidocaine is a local anesthetic with anti-inflammatory effect. When used to treat cystitis, it is most potent when administered in its alkalinized form, which allows better penetration of bladder epithelium. Thus, the disclosed compositions may be formulated at a pH that supports the formation of alkalized lidocaine (e.g., a pH of 7.4 to 8.0). While not being bound by theory, it is believed that formulating lidocaine at such a pH can offer advantages such as better penetrating ability and longer pain-relieving effect as compared to a formulation of neutral lidocaine. See, e.g., Henry et al., Urology 85: 1025-1033 (2015); Henry et al., J Urol. 165: 1900-1903 (2001).

The disclosed compositions may exhibit good stability under ambient conditions (e.g., 25° C. and 60% relative humidity) and are suitable for long-term storage (e.g., at least 3 months).

### Definitions

Technical and scientific terms used herein have the meanings commonly understood by one of ordinary skill in the art of pharmaceutical formulations to which the present disclosure pertains, unless otherwise defined. Reference is made herein to various methodologies known to those of ordinary skill in the art. Suitable materials and/or methods known to those of ordinary skill in the art can be utilized in carrying out the present disclosure. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the terms “comprising,” “including,” and “containing” are used expansively to mean that the described compositions, methods, or kits include at least the stated elements, and may include other elements that are not specified.

As used herein, the term “about” means that the number or range so modified is not limited to the exact number or range set forth but encompass values around the stated number or range as will be understood by persons of ordinary skill in the art depending on the context in which the number or range is used. Unless otherwise apparent from the context or convention in the art, “about” means up to plus or minus 10% of the particular term.

As used herein, “subject” denotes any mammal, including humans. For example, a subject may be suffering from or at risk of developing a condition that can be diagnosed, treated or prevented with a pharmaceutical composition as described herein, or may be taking a pharmaceutical composition described herein for other purposes.

The terms “administer,” “administration,” or “administering” as used herein refer to (1) providing, giving, dosing and/or prescribing, such as by a health professional or his or her authorized agent or under his direction, or the subject, and (2) putting into, taking or consuming, such as by a health professional or the subject.

The terms “treat,” “treating,” or “treatment,” as used herein, include alleviating, abating or ameliorating a disease or condition, whether or not the disease or condition is considered to be “cured” or “healed” and whether or not all symptoms are resolved. The terms also include reducing, slowing, or preventing progression of a disease or condition, impeding or preventing an underlying mechanism of a disease or condition, and achieving a therapeutic and/or prophylactic benefit vis-à-vis a disease or condition.

As used herein, the phrase “therapeutically effective amount” refers to a dose that provides the specific pharmacological effect for which the drug is administered in a subject in need of such treatment. It is emphasized that a therapeutically effective amount may not always be effective in treating the conditions described herein, even though such dose is deemed to be a therapeutically effective amount by those of skill in the art. For convenience only, exemplary doses and therapeutically effective amounts are provided below with reference to adult human subjects. Those skilled in the art can adjust such amounts in accordance with standard practices as needed to treat a specific subject and/or condition/disease.

The term “pharmaceutically acceptable salt” as used herein, refers to pharmaceutically acceptable organic or inorganic salts of the referenced compound. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts.

Exemplary pharmaceutical compositions, methods of making them, and methods of using them, as well as specific illustrative embodiments, are discussed in more detail below.

### Compositions

As noted above, provided herein are aqueous pharmaceutical compositions that include lidocaine, hyaluronic acid, and chondroitin sulphate, or a pharmaceutically acceptable salt of any one or more thereof, and water (e.g., purified water).

For example, a composition as described herein may comprise lidocaine or a pharmaceutically acceptable salt thereof. In some embodiments, the lidocaine or pharmaceutically acceptable salt thereof is lidocaine hydrochloride. The lidocaine (or pharmaceutically acceptable salt thereof, such as lidocaine hydrochloride) can be present in any suitable amount, such as any therapeutically effective amount for treating a disease described herein (e.g., cystitis).

In some embodiments, a composition as described herein comprises from about 0.05% to about 0.6% w/v of lidocaine (or a pharmaceutically acceptable salt thereof, such as lidocaine hydrochloride), including from 0.05% to 0.6% w/v, such as about 0.05% w/v, about 0.075% w/v, about 0.1% w/v, about 0.15% w/v, about 0.2% w/v, about 0.25% w/v, about 0.3% w/v, about 0.35% w/v, about 0.4% w/v, about 0.45% w/v, about 0.5% w/v, about 0.55% w/v, or about 0.6% w/v, or any value therebetween. In some embodiments, a composition as described herein comprises about 0.4% w/v lidocaine (or a pharmaceutically acceptable salt thereof, such as lidocaine hydrochloride), such as 0.4% w/v lidocaine hydrochloride. In some embodiments, a composition as described herein comprises about 0.2% w/v lidocaine (or a pharmaceutically acceptable salt thereof, such as lidocaine hydrochloride), such as 0.2% w/v lidocaine hydrochloride.

A pharmaceutical composition as described herein also comprises hyaluronic acid or a pharmaceutically acceptable salt thereof. In some embodiments, the hyaluronic acid or pharmaceutically acceptable salt thereof is sodium hyaluronate. In some embodiments, the hyaluronic acid or pharmaceutically acceptable salt thereof has a weight-average molecular weight of from about 50 to about 500 kDa, such as about 50 kDa, about 100 kDa, about 200 kDa, about 300 kDa, about 400 kDa, or about 500 kDa, or any value therebetween.

The hyaluronic acid (or pharmaceutically acceptable salt thereof, such as sodium hyaluronate) can be present in any suitable amount, such as any therapeutically effective amount for treating a disease described herein (e.g., cystitis). In some embodiments, a composition as described herein comprises from about 0.25% to about 3.0% w/v of hyaluronic acid (or pharmaceutically acceptable salt thereof, such as sodium hyaluronate), including from 0.25% to 3.0% w/v, such as about 0.25% w/v, about 0.5% w/v, about 0.75% w/v, about 1.0% w/v, about 1.5% w/v, about 2.0% w/v, about 2.5% w/v, or about 3.0% w/v, or any value therebetween. In some embodiments, a composition as described herein comprises about 1.5% w/v of hyaluronic acid or pharmaceutically acceptable salt thereof, such as sodium hyaluronate, including 1.5% w/v. In some embodiments, a composition as described herein comprises 1.6% w/v of hyaluronic acid or a pharmaceutically acceptable salt thereof, such as sodium hyaluronate. In some embodiments, a composition as described herein comprises about 0.75% w/v of hyaluronic acid or pharmaceutically acceptable salt thereof, such as sodium hyaluronate, including 0.75% w/v. In some embodiments, a composition as described herein comprises 0.8% w/v of hyaluronic acid or a pharmaceutically acceptable salt thereof, such as sodium hyaluronate.

A pharmaceutical composition as described herein also comprises chondroitin sulphate or a pharmaceutically acceptable salt thereof. In some embodiments, the chondroitin sulphate or pharmaceutically acceptable salt thereof is chondroitin sulphate sodium. In some embodiments, the chondroitin sulphate or pharmaceutically acceptable salt thereof (such as chondroitin sulphate sodium) has a weight-average molecular weight of from about 1 to about 200 kDa, such as about 1, about 50 kDa, about 100 kDa, about 150 kDa, or about 200 kDa, or any value therebetween.

The chondroitin sulphate (or pharmaceutically acceptable salt thereof such as chondroitin sulphate sodium) can be present in any suitable amount, such as any therapeutically effective amount for treating a disease described herein (e.g., cystitis). In some embodiments, a composition as described herein comprises from about 0.3% to about 3.5% w/v of chondroitin sulphate (or pharmaceutically acceptable salt thereof such as chondroitin sulphate sodium), including from 0.3% to 3.5% w/v, such as about 0.3% w/v, about 0.5% w/v, about 0.75% w/v, about 1.0% w/v, about 1.5% w/v, about 2.0% w/v, about 2.5% w/v, about 3.0% w/v, or about 3.5% w/v, or any value therebetween. In some embodiments, a composition as described herein comprises about 2.0% w/v of chondroitin sulphate (or pharmaceutically acceptable salt thereof such as chondroitin sulphate sodium), including 2.0% w/v. In some embodiments, a composition as described herein comprises about 1.0% w/v of chondroitin sulphate (or pharmaceutically acceptable salt thereof such as chondroitin sulphate sodium), including 1.0% w/v. In some embodiments, a composition as described herein comprises 2.0% w/v chondroitin sulphate sodium. In some embodiments, a composition as described herein comprises 1.0% w/v chondroitin sulphate sodium.

As noted above, formulating a composition to promote formation of alkalized lidocaine (the ionized form of lidocaine) can be advantageous in the context of providing pain relief to a subject in need thereof (e.g., a subject suffering from cystitis). In this regard, an aqueous pharmaceutical composition having a pH of 6.0 or less typically does not provide a sufficient amount of ionized lidocaine to achieve this beneficial effect. Accordingly, an aqueous pharmaceutical composition as described herein typically is prepared to have a pH of from about 6.0 to about 8.5, including from 6.0 to 8.5, such as from about 7.3 to about 7.5, including from 7.3 to 7.5, such as about 7.4, or 7.4. In some embodiments, a composition as described herein has a pH of about 7.4. In some embodiments, a composition as described herein has a pH of 7.4.

In some embodiments, a composition as described herein further comprise a pH adjusting agent. Examples of suitable pH adjusting agents include meglumine, sodium hydroxide (NaOH), and sodium bicarbonate. In some embodiments, the pH adjusting agent is meglumine. In some embodiments, the pH adjusting agent is NaOH. NaOH may be used at a concentration of 0.1 to 0.2 M, such that it contributes a negligible amount of sodium ions to the composition, and does not cause irritation or exacerbate pain or other symptoms. In some embodiments, the pH adjusting agent is sodium bicarbonate. As discussed below, however, in some embodiments a composition as described herein does not include a bicarbonate salt. Thus, in some embodiments the pH adjusting agent is not sodium bicarbonate.

When present, the pH adjusting agent can be present in any suitable amount, such as any amount effective to achieve a target pH of the composition. Thus, for example, the pH adjusting agent can be present in an amount effective to achieve a pH of the composition of from about 6.0 to about 8.5, including from 6.0 to 8.5, including from about 7.3 to about 7.5, including from 7.3 to 7.5, such as about 7.4, or 7.4.

A composition as described herein does not rely on or require a buffering agent to maintain pH at a target value or within a target range. Thus, in some embodiments a composition as described herein does not include a buffering agent, e.g., the composition is prepared without adding a buffer, such that the composition is free of a buffer. In some embodiments, a composition as described herein does not include a buffering agent in an amount effective to buffer the pH of the composition. In such embodiments small amounts of a species that could act as a buffer may be present in the composition, either accidentally or intentionally. In some embodiments, a species that could in theory act as a buffer is present in an amount <0.1 mg/mL. In some embodiments, a species that could in theory act as a buffer is present in an amount <0.05 mg/mL. In some embodiments, a species that could in theory act as a buffer is present in an amount <0.01 mg/mL.

Examples of buffering agents (e.g., species that could in theory act as a buffer) that may be excluded from a composition as described herein include one or more or all of the following: a phosphate salt, a carbonic acid, a bicarbonate salt, an acetic acid, an acetate salt, a silicate salt, a citric acid, and a citrate salt. In some embodiments, a composition as described herein does not include any of the following buffering agents: a phosphate salt, a carbonic acid, a bicarbonate salt, an acetic acid, an acetate salt, a silicate salt, a citric acid, and a citrate salt. Thus, in some embodiments, all of these are absent from the composition. In some embodiments, the total amount of any these present is <0.1 mg/mL, including <0.05 mg/mL, or <0.01 mg/mL. In specific embodiments, a composition as described herein does not include a phosphate buffer. In other specific embodiments, a composition as described herein does not include ≥0.1 mg/ml of a phosphate buffer, or does not include ≥0.05 mg/mL of a phosphate buffer, or does not include ≥0.01 mg/mL of a phosphate buffer. In this context, the term “phosphate buffer” includes any phosphate salts that can act as a buffer in an aqueous composition.

The following are disclosed as specific illustrative embodiments of aqueous pharmaceutical compositions as described herein.

In some embodiments, an aqueous pharmaceutical composition as described herein comprises:
(i) 0.05% to 0.6% w/v of lidocaine or a pharmaceutically acceptable salt thereof;
(ii) 0.25% to 3.0% w/v of hyaluronic acid or a pharmaceutically acceptable salt thereof;
(iii) 0.3% to 3.5% w/v of chondroitin sulphate or a pharmaceutically acceptable salt thereof, and
(iv) a pH adjusting agent in an amount effective to achieve a pH of the composition of from 6.0 to 8.5.

In some embodiments, the composition does not include a buffering agent. In some embodiments, the composition does not include a buffering agent in an amount effective to buffer the pH of the composition.

In some embodiments, provided herein are aqueous pharmaceutical compositions, comprising:
(i) 0.05% to 0.6% w/v of lidocaine or a pharmaceutically acceptable salt thereof;
(ii) 0.25% to 3.0% w/v of hyaluronic acid or a pharmaceutically acceptable salt thereof;
(iii) 0.3% to 3.5% w/v of chondroitin sulphate or a pharmaceutically acceptable salt thereof, and
(iv) a pH adjusting agent in an amount effective to achieve a pH of the composition of from 6.0 to 8.5,
wherein the composition does not include a buffering agent in an amount effective to buffer the pH of the composition.

In some embodiments, an aqueous pharmaceutical composition as described herein comprises:
(i) 0.4% w/v of lidocaine hydrochloride,
(ii) 1.6% w/v of sodium hyaluronate,
(iii) 2% chondroitin sulphate sodium, and
(iv) a pH adjusting agent selected from meglumine and NaOH,
wherein the composition does not include a buffering agent in an amount effective to buffer the pH of the composition.

In some embodiments, an aqueous pharmaceutical composition as described herein comprises:
(i) 0.2% w/v of lidocaine hydrochloride,
(ii) 0.8% w/v of sodium hyaluronate,
(iii) 1% chondroitin sulphate sodium, and
(iv) a pH adjusting agent selected from meglumine and NaOH,
wherein the composition does not include a buffering agent in an amount effective to buffer the pH of the composition.

In accordance with specific embodiments of any of the foregoing embodiments, the composition does not include one or more or all of the following buffering agents: a phosphate salt, a carbonic acid, a bicarbonate salt, an acetic acid, an acetate salt, a silicate salt, a citric acid, and a citrate salt. In accordance with specific embodiments of any of the foregoing embodiments, the composition does not include any of the following buffering agents: a phosphate salt, a carbonic acid, a bicarbonate salt, an acetic acid, an acetate salt, a silicate salt, a citric acid, and a citrate salt.

In accordance with specific embodiments of any of the foregoing embodiments, the composition may comprise about 4 mg/mL lidocaine hydrochloride, including 4 mg/mL lidocaine hydrochloride. Alternatively, the composition may comprise about 2 mg/mL lidocaine hydrochloride, including 2 mg/mL lidocaine hydrochloride.

In accordance with specific embodiments of any of the foregoing embodiments, the composition comprises about 16 mg/mL sodium hyaluronate, including 16 mg/mL sodium hyaluronate. Alternatively, the composition may comprise about 8 mg/mL sodium hyaluronate, including 8 mg/mL sodium hyaluronate.

In accordance with specific embodiments of any of the foregoing embodiments, the composition comprises about 20 mg/mL chondroitin sulphate sodium, including 20 mg/mL chondroitin sulphate sodium. Alternatively, the composition may comprise about 10 mg/mL chondroitin sulphate sodium, including 10 mg/mL chondroitin sulphate sodium.

In accordance with specific embodiments of any of the foregoing embodiments, the composition comprises about 4 mg/mL lidocaine hydrochloride, about 16 mg/mL sodium hyaluronate, and about 20 mg/mL chondroitin sulphate sodium. In accordance with further specific embodiments of any of the foregoing embodiments, the composition comprises 4 mg/mL lidocaine hydrochloride, 16 mg/mL sodium hyaluronate, and 20 mg/mL chondroitin sulphate sodium.

Alternatively, in accordance with other specific embodiments of any of the foregoing embodiments, the composition comprises about 2 mg/mL lidocaine hydrochloride, about 8 mg/mL sodium hyaluronate, and about 10 mg/mL chondroitin sulphate sodium. In accordance with further specific embodiments of the foregoing embodiments, the composition comprises 2 mg/mL lidocaine hydrochloride, 8 mg/mL sodium hyaluronate, and 10 mg/mL chondroitin sulphate sodium.

In accordance with any of the foregoing embodiments, an aqueous pharmaceutical composition as described herein may be prepared as a sterile solution formulated for instillation in the urogenital tract or bladder.

In accordance with any of the foregoing embodiments, an aqueous pharmaceutical composition as described herein may be packaged into unit dosage forms (unit doses) containing a pharmaceutically effective dose of lidocaine hydrochloride. In some embodiments, the aqueous pharmaceutical composition is packaged in a unit dose form comprising 200 mg lidocaine hydrochloride in 50 mL. In some embodiments, the aqueous pharmaceutical composition is packaged in a unit dose form comprising 100 mg lidocaine hydrochloride in 50 mL.

### Stability

The aqueous pharmaceutical compositions described herein exhibit good stability against the formation of impurities, including degradation product(s). For example, a composition as described herein can be stable for at least 1 month when stored at about 25° C. and 60% relative humidity, at about 30° C. and 75% relative humidity, or at about 40° C. and 75% relative humidity, including being stable for at least 2 months or at least 3 months when stored under one or those conditions. In some embodiments, an aqueous pharmaceutical composition as described herein is stable for at least 1 month when stored at 25° C. and 60% relative humidity. In some embodiments, an aqueous pharmaceutical composition as described herein is stable for at least 2 months when stored at 25° C. and 60% relative humidity. In some embodiments, an aqueous pharmaceutical composition as described herein is stable for at least 3 months when stored at 25° C. and 60% relative humidity.

In some embodiments, stability of an aqueous composition as described herein can be assessed by the amount of lidocaine degradation product(s) present in the composition. For example, in some embodiments, a composition containing less than about 0.5 wt % (e.g., less than 0.5 wt %) degradation products may be considered to be stable. In some embodiments, the amount of lidocaine degradation product(s) present in the composition after storage for 3 months at 25° C. and 60% relative humidity is less than about 0.4 wt %, including less than 0.4 wt %, such as less than about 0.3 wt %, such as less than 0.3 wt %, such as less than about 0.2 wt %, such as less than 0.2 wt %, less than about 0.1 wt %, or less than about 0.1 wt %. In some embodiments, the amount of lidocaine degradation product(s) in the pharmaceutical composition after storage for 3 months at 30° C. and 75% relative humidity is less than about 0.4 wt %, including less than 0.4 wt %, such as less than about 0.3 wt %, such as less than 0.3 wt %, such as less than about 0.2 wt %, such as less than 0.2 wt %, less than about 0.1 wt %, or less than about 0.1 wt %. In some embodiments, the amount of lidocaine degradation product(s) in the pharmaceutical composition after storage for 3months at 40° C. and 75% relative humidity is less than about 0.5 wt %, including less than 0.5 wt %.

Additionally or alternatively, in some embodiments, stability of an aqueous composition as described herein can be assessed by the change in pH value. For example, a composition having a change in pH of 0.3 or less may be considered to be stable. In some embodiments, the pH of a composition as described herein changes by about 0.3 or less, including 0.3 or less, about 0.2 or less, 0.2 or less, about 0.1 or less, or 0.1 or less, after storage for 3 months at 25° C. and 60% relative humidity. In some embodiments, the pH of a composition as described herein changes by about 0.3 or less, including 0.3 or less, about 0.2 or less, 0.2 or less, about 0.1 or less, or 0.1 or less, after storage for 3 months at 30° C. and 75% relative humidity. In some embodiments, the pH of a composition as described herein changes by about 0.3 or less, including 0.3 or less, about 0.2 or less, 0.2 or less, about 0.1 or less, or 0.1 or less, after storage for 3 months at 40° C. and 75% relative humidity.

### Therapeutic Methods/Uses

As noted above, the aqueous pharmaceutical compositions described herein can be used in treatment methods, such as methods of treating cystitis. Such methods comprise administering a composition as described herein to a subject in need thereof at a therapeutically effective dose. For example, the subject may be a human subject suffering from cystitis (e.g., cystitis associated with an infection, chemotherapy-induced cystitis, radiation-induced cystitis, or interstitial cystitis). The composition may be administered by any suitable route of administration. In some embodiments, the composition is administered by instillation into the urogenital tract or bladder.

Administration of a composition as described herein to a subject in need thereof may achieve therapeutic effects, such as one or more of a reduction in nociceptive pain score and a reduction in bladder pressure after administration. As noted above, administration of a composition as described herein can be associated with fewer side effects than a composition comprising a buffer, due to avoidance of the risks of irritation and pain that can be associated with ions present in a buffered solution.

### EXAMPLES

The following examples are included as illustrative of the compositions described herein. These examples are in no way intended to limit the scope of the disclosure. Other aspects of the disclosure will be apparent to those skilled in the art to which the disclosure pertains.

### Example 1: Preparation of Aqueous Pharmaceutical Compositions

Aqueous pharmaceutical compositions having the components set forth in Table 1 below were prepared as outlined below. Under nitrogen purging, buffering agent (e.g., Na₂HPO₄·2H₂O and NaH₂PO₄·H₂O) (if being used) was added to the purified water, followed by lidocaine hydrochloride and chondroitin sulphate sodium. Nitrogen purging was stopped, and sodium hyaluronate was added. The pH of the resulting solution was adjusted to the target pH (e.g., 7.4) using a pH adjusting agent (e.g., meglumine or NaOH), and additional purified water was added to achieve the final volume.

TABLE 1

| | 1 | 2 | | |
|---|---|---|---|---|
| Formulation | (Comparative) | (Comparative) | 3 | 4 |
| Ingredients | | | | |
| Lidocaine HCl | 4 | 4 | 4 | 4 |
| (mg/mL) | | | | |
| Sodium Hyaluronate | 16 | 16 | 16 | 16 |
| (mg/mL) | | | | |
| Chondroitin | 20 | 20 | 20 | 20 |
| sulphate | | | | |
| sodium (mg/mL) | | | | |
| Na₂HPO₄•2H₂O | 0.27 | 0.27 | — | — |
| (mg/mL) | | | | |
| NaH₂PO₄•H₂O | 0.07 | 0.07 | — | — |
| (mg/mL) | | | | |
| Meglumine | q.s. to | — | q.s. to | — |
| (pH adjusting agent) | pH 7.4 | | pH 7.4 | |
| NaOH | — | q.s. to | — | q.s. to |
| (pH adjusting agent) | | pH 7.4 | | pH 7.4 |
| pH of | 7.40 | 7.40 | 7.40 | 7.40 |
| final composition | | | | |
| Observation | Clear | Clear | Clear | Clear |
| | solution | solution | solution | solution |

### Example 2: Stability of Aqueous Pharmaceutical Compositions

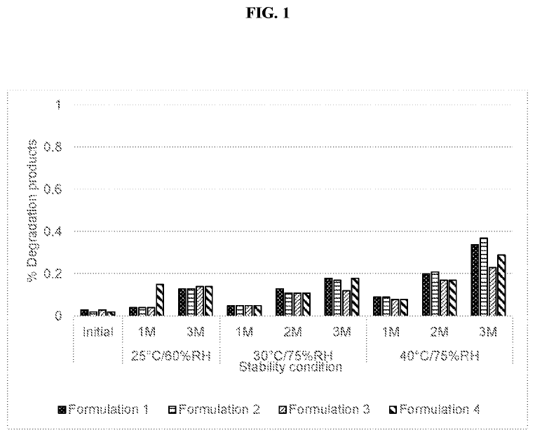

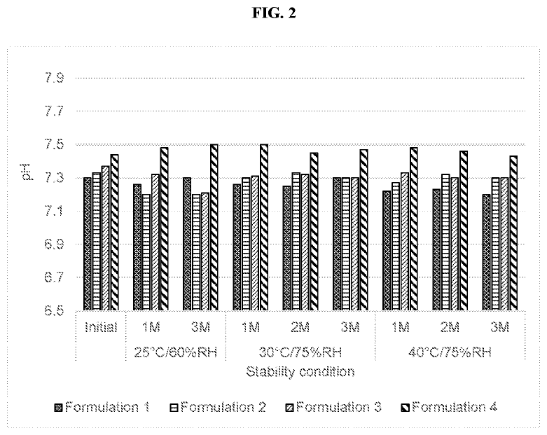

Comparative Formulations 1 and 2 and Formulations 3 and 4 prepared as described above were stored under various conditions (25° C. and 60% relative humidity, 30° C. and 75% relative humidity, and 40° C. and 75% relative humidity, respectively) for up to 3 months. Stability was assessed visually, and with regard to formation of lidocaine degradation product(s) and change in pH. All formulations remained clear and colorless after three months storage under all conditions. As reported in FIG. 1 and FIG. 2, both Formulations 3 and 4 showed formation of less than 0.3% w/w lidocaine degradation products after storage for up to three months at 25° C. and 60% relative humidity or 30° C. and 75% relative humidity, and less than 0.4% w/w lidocaine degradation products after storage for three months at 40° C. and 75% relative humidity, and changes in pH of less than 0.3 after three months storage under all conditions.

These results surprisingly show that the pH of a composition as described herein is stable over time even without the presence of a buffer. These results also surprisingly show that the stability of a composition as described herein against the formation of degradation products is at least comparable to a composition formulated with a buffer (e.g., Formulations 1 and 2), e.g., that the compositions are stable without the need for a buffer.

### Example 3: Additional Aqueous Pharmaceutical Compositions

Aqueous pharmaceutical compositions having the components set forth in Table 2 below were prepared as outlined above.

TABLE 2

| Formulation | 5 | 6 | 7 | 8 |
|---|---|---|---|---|
| Ingredients | | | | |
| Lidocaine HCl | 4 | 4 | 2 | 2 |
| (mg/mL) | | | | |
| Sodium Hyaluronate | 16 | 16 | 8 | 8 |
| (mg/mL) | | | | |
| Chondroitin sulphate | 20 | 20 | 10 | 10 |
| sodium (mg/mL) | | | | |
| Meglumine | q.s. to | q.s. to | q.s. to | — |
| (pH adjusting agent) | pH 6.5 | pH 8.5 | pH 7.4 | |
| NaOH | — | — | — | q.s. to |
| (pH adjusting agent) | | | | pH 7.4 |
| pH of final composition | 6.40 | 8.30 | 7.40 | 7.40 |
| Observation | Clear | Clear | Clear | Clear |
| | solution | solution | solution | solution |

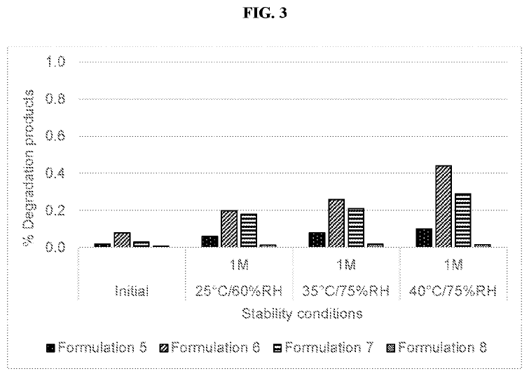

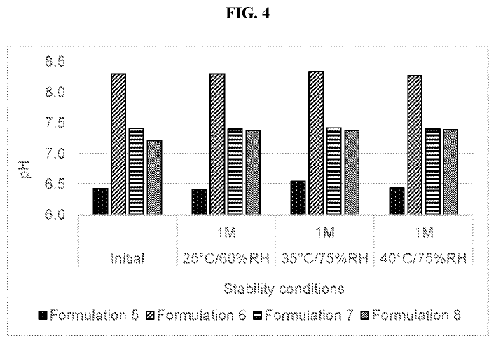

Formulations 5 to 8 were prepared as described above and stored under various conditions (25° C. and 60% relative humidity, 30° C. and 75% relative humidity, and 40° C. and 75% relative humidity, respectively) for 1 month. Stability was assessed visually, and with regard to formation of lidocaine degradation product(s) and change in pH. All formulations remained clear and colorless after one month storage under all conditions. As reported in FIG. 3 and FIG. 4, Formulations 5, 7 and 8 showed formation of less than 0.3% w/w lidocaine degradation products after storage for one month at 25° C. and 60% relative humidity or 30° C. and 75% relative humidity, and less than 0.4% w/w lidocaine degradation products after storage for one month at 40° C. and 75% relative humidity, and a pH change of less than 0.3 after one month storage under all conditions.

Results for Formulation 6, which was formulated with a pH of 8.3, are set forth in FIG. 3 and FIG. 4. As seen in the figures, Formulation 6 showed a change in pH of less than 0.3 after one month storage under all conditions. Formulation 6 showed formation of less than 0.3% w/w lidocaine degradation products after one month storage at 25° C. and 60% relative humidity, or at 30° C. and 75% relative humidity, and formation of less than 0.5% w/w lidocaine degradation products after one month storage at 40° C. and 75% relative humidity. These results indicate that formulations prepared at pH>8.5 may not be as stable against the formation of lidocaine degradation products as formulations prepared at pH<8.5 (e.g., at pH from 6.0 to 8.5).

What is claimed is:

1. An aqueous pharmaceutical composition comprising:
(i) 0.05% to 0.6% w/v of lidocaine or a pharmaceutically acceptable salt thereof;
(ii) 0.25% to 3.0% w/v of hyaluronic acid or a pharmaceutically acceptable salt thereof;
(iii) 0.3% to 3.5% w/v of chondroitin sulphate or a pharmaceutically acceptable salt thereof, and
(iv) a pH adjusting agent in an amount effective to achieve a pH of the composition of from 6.0 to 8.5,
wherein the composition does not include a buffering agent in an amount effective to buffer the pH of the composition.

2. The composition of claim 1, wherein the composition does not include one or more or all of the following buffering agents: a phosphate salt, a carbonic acid, a bicarbonate salt, an acetic acid, an acetate salt, a silicate salt, a citric acid, and a citrate salt.

3. The composition of claim 1, wherein the composition does not include any of the following buffering agents: a phosphate salt, a carbonic acid, a bicarbonate salt, an acetic acid, an acetate salt, a silicate salt, a citric acid, and a citrate salt.

4. The composition of any one of the preceding claims, wherein the hyaluronic acid or pharmaceutically acceptable salt thereof, has a weight-average molecular weight of from about 50 to about 500 kDa.

5. The composition of any one of the preceding claims, wherein the chondroitin sulphate or pharmaceutically acceptable salt thereof has a weight-average molecular weight of from about 1 to about 200 kDa.

6. The composition of any one of the preceding claims, wherein the lidocaine or pharmaceutically acceptable salt thereof is lidocaine hydrochloride.

7. The composition of any one of the preceding claims, wherein the hyaluronic acid or pharmaceutically acceptable salt thereof is sodium hyaluronate.

8. The composition of any one of the preceding claims, wherein the chondroitin sulphate or pharmaceutically acceptable salt is chondroitin sulphate sodium.

9. The composition of any one of the preceding claims, wherein the pH adjusting agent is selected from meglumine and NaOH.

10. The composition of any one of the preceding claims, comprising:
(i) 0.4% w/v of lidocaine hydrochloride,
(ii) 1.6% w/v of sodium hyaluronate,
(iii) 2% w/v of chondroitin sulphate sodium, and
(iv) a pH adjusting agent selected from meglumine and NaOH.

11. The composition of any one of the preceding claims, wherein the pharmaceutical composition comprises about 4 mg/mL lidocaine hydrochloride, about 16 mg/mL sodium hyaluronate, and about 20 mg/mL chondroitin sulphate sodium (CS).

12. The composition of any one of the preceding claims, wherein the composition is packaged in a unit dose form comprising 200 mg lidocaine hydrochloride in 50 mL.

13. The composition of any one of claims 1-9, comprising:
(i) 0.2% w/v of lidocaine hydrochloride,
(ii) 0.8% w/v of sodium hyaluronate,
(iii) 1% w/v of chondroitin sulphate sodium, and
(iv) a pH adjusting agent selected from meglumine and NaOH.

14. The composition of any one of claims 1-9 and 13, wherein the pharmaceutical composition comprises about 2 mg/mL lidocaine hydrochloride, about 8 mg/mL sodium hyaluronate, and about 10 mg/mL chondroitin sulphate sodium (CS).

15. The composition of any one of claims 1-9 and 13-14, wherein the composition is packaged in a unit dose form comprising 100 mg lidocaine hydrochloride in 50 mL.

16. The composition of any one of the preceding claims, having a pH from 7.3 to 7.5.

17. The composition of any one of the preceding claims, having a pH of about 7.4.

18. The composition of any one of the preceding claims, comprising an amount of pH adjusting agent effective to adjust the pH to about 7.4.

19. The composition of any one of the preceding claims, wherein the composition is a sterile solution formulated for instillation in the urogenital tract or bladder.

20. The composition of any one of the preceding claims, wherein the composition is stable for at least 3 months when stored at 25° C. and 60% relative humidity.

21. The composition of any one of the preceding claims, wherein, after storage for 3 months at 25° C. and 60% relative humidity, the amount of lidocaine degradation products in the pharmaceutical composition is less than 0.4 wt %.

22. The composition of any one of the preceding claims, wherein the composition is stable for at least 3 months when stored at 30° C. and 75% relative humidity.

23. The composition of any one of the preceding claims, wherein, after storage for 3 months at 30° C. and 75% relative humidity, the amount of lidocaine degradation products in the pharmaceutical composition is less than 0.4 wt %.

24. The composition of any one of the preceding claims, wherein the composition is stable for at least 3 months when stored at 40° C. and 75% relative humidity.

25. The composition of any one of the preceding claims, wherein, after storage for 3 months at 40° C. and 75% relative humidity, the amount of lidocaine degradation products in the pharmaceutical composition is less than 0.5 wt %.

26. The composition of any one of the preceding claims, wherein, after storage for 3 months at 25° C. and 60% relative humidity, the pH of the pharmaceutical composition differs by no more than about 0.3 from the original pH.

27. A method of treating cystitis, comprising administering a pharmaceutical composition according to any one of the preceding claims to a subject in need thereof.

28. The method of claim 27, wherein the pharmaceutical composition is administered by instillation into the urogenital tract or bladder.

29. The method of any one of claims 27-28, wherein the subject has a reduction in nociceptive pain score after administration of the pharmaceutical composition.

30. The method of any one of claims 27-29, wherein the subject has a lower bladder pressure after administration of the pharmaceutical composition.

31. The method of any one of claims 27-30, wherein the cystitis is interstitial cystitis.

32. An aqueous pharmaceutical composition according to any one of claims 1-26, for use in treating cystitis.

33. The pharmaceutical composition for use of claim 32, wherein the cystitis is interstitial cystitis.

34. Use of the aqueous pharmaceutical composition according to any one of claims 1-26, for the manufacture of a medicament for treating cystitis.

35. The use of claim 34, wherein the cystitis is interstitial cystitis.
